(19) United States
(12) Patent Application Publication
(10) Pub. No.: US 2025/0162961 A1
(43) Pub. Date: May 22, 2025

(54) SYSTEM AND METHODS FOR PRODUCING METHANOL USING CARBON DIOXIDE

(71) Applicant: Bechtel Energy Technologies & Solutions, Inc., Houston, TX (US)

(72) Inventors: Thomas M. Jones, Sugar Land, TX (US); Faisal Mohmand, Sugar Land, TX (US)

(73) Assignee: Bechtel Energy Technologies & Solutions, Inc., Houston, TX (US)

(21) Appl. No.: 18/862,098

(22) PCT Filed: Dec. 1, 2023
(86) PCT No.: PCT/US23/82147

§ 371 Date: Oct. 31, 2024

Related U.S. Application Data
(60) Provisional application No. 63/430,210, filed on Dec. 5, 2022.

(51) Int. Cl. C07C 1/12; C01B 3/34; C07C 29/80; C25B 1/04; C25B 15/08

(52) U.S. Cl. CPC C07C 1/12; C01B 3/34; C07C 29/80; C25B 1/04; C25B 15/081; C01B 2203/061

(57) ABSTRACT

Systems and methods for producing methanol using syngas, which is a primarily a mixture of hydrogen and carbon monoxide, hydrogen and a carbon dioxide by-product that significantly reduce carbon dioxide emissions and/or sequestration. The syngas may be produced, for example, by an autothermal reactor, a steam methane reformer, or a gasifier. The hydrogen may be produced by an electrolyzer.

### RELATED APPLICATIONS

This application claims priority to U.S. Provisional Application No. 63/430,210, filed Dec. 5, 2022, which is incorporated herein by reference.

### FIELD OF THE DISCLOSURE

The present disclosure relates to systems and methods for producing methanol using carbon dioxide. More particularly, the systems and methods use syngas, which is a primarily a mixture of hydrogen and carbon monoxide, hydrogen and a carbon dioxide by-product to produce methanol and significantly reduce carbon dioxide emissions and/or sequestration.

### BACKGROUND

Conventional methods for producing methanol are based on fossil fuels such as natural gas (NG) and naphtha in steam methane reformers, autothermal reformers, and the like. These methods produce carbon dioxide (CO2) and other greenhouse gases that are either emitted to the atmosphere or must be captured and sequestered.

Syngas may be produced by steam reforming or partial oxidation of hydrocarbons. In either case, CO2 is a by-product that must be emitted to the atmosphere or captured and sequestered. The feedstocks for steam reforming include natural gas, natural gas liquids, and naphtha, which are generally converted by catalytic steam reforming to a raw synthesis gas consisting of hydrogen (H2) and carbon monoxide (CO). The raw synthesis gas is then further processed depending on the desired final products. In the case of pure hydrogen, the process includes such features as the catalytic conversion of the CO and a pressure swing adsorption unit, in which all impurities are removed in a single step. The feedstock for partial oxidation is heavy oil and includes anything from residual oil to asphalt and coal, which are partially combusted with oxygen (O2) in a non-catalytic partial oxidation to produce a raw synthesis gas consisting of H2 and CO. The syngas may be further processed to produce methanol or other saleable products.

Electrolysis is a promising option for carbon-free hydrogen production from renewable and nuclear resources. Electrolysis uses electricity to split water into H2 and O2. This reaction takes place in a unit called an electrolyzer, which can range in size from small, appliance-size equipment that is well-suited for small-scale distributed hydrogen production to large-scale, central production facilities that could be tied directly to renewable or other non-greenhouse-gas-emitting forms of electricity production.

### BRIEF DESCRIPTION OF THE DRAWINGS

The detailed description is described below with reference to the accompanying drawing, in which like elements are referenced with like reference numbers, and in which:

FIG. 1 is a schematic diagram illustrating one embodiment of a system for producing methanol using syngas produced by an autothermal reformer reactor, H2 produced by an electrolyzer and a CO2 by-product.

FIG. 2 is a schematic diagram illustrating one embodiment of a system for producing methanol using syngas produced by a steam methane reformer, H2 produced by an electrolyzer and a CO2 by-product.

FIG. 3 is a schematic diagram illustrating one embodiment of a system for producing methanol using syngas produced by a gasifier, H2 produced by an electrolyzer and a CO2 by-product.

### DETAILED DESCRIPTION OF THE ILLUSTRATIVE EMBODIMENTS

The subject matter of the present disclosure is described with specificity, however, the description itself is not intended to limit the scope of the disclosure. The subject matter thus, might also be embodied in other ways, to include different structures, steps and/or combinations similar to and/or fewer than those described herein, in conjunction with other present or future technologies. Although the term “step” may be used herein to describe different elements of methods employed, the term should not be interpreted as implying any particular order among or between various steps herein disclosed unless otherwise expressly limited by the description to a particular order. Other features and advantages of the disclosed embodiments will be or will become apparent to one of ordinary skill in the art upon examination of the following figures and detailed description. It is intended that all such additional features and advantages be included within the scope of the disclosed embodiments. Further, the illustrated figures and dimensions described herein are only exemplary and are not intended to assert or imply any limitation with regard to the environment, architecture, design, or process in which different embodiments may be implemented. To the extent that temperatures and pressures are referenced in the following description, those conditions are merely illustrative and are not meant to limit the disclosure. All streams described herein are carried by physical lines.

The systems and methods disclosed herein integrate conventional processes that produce syngas and H2, which are used with a CO2 by-product as feedstock in a methanol reactor to produce methanol and significantly reduce CO2 emissions and/or sequestration. The methanol product may be used as a chemical feedstock or as fuel.

In one embodiment, the present disclosure includes a system for producing methanol, which comprises: i) a methanol reactor for producing a methanol reactor output stream, the methanol reactor comprising syngas from a syngas stream, hydrogen from a hydrogen stream and carbon dioxide from a carbon dioxide stream; ii) a separator for separating the methanol reactor output stream into a vapor stream and a liquid stream; and iii) a distillation column for separating the liquid stream into a methanol product stream and a water stream.

In another embodiment, the present disclosure includes a method for producing methanol, which comprises: i) introducing a syngas stream, a hydrogen stream and a carbon dioxide stream into a methanol reactor for producing a methanol reactor output stream; ii) separating the methanol reactor output stream into a vapor stream and a liquid stream; and iii) separating the liquid stream into a methanol product stream and a water stream.

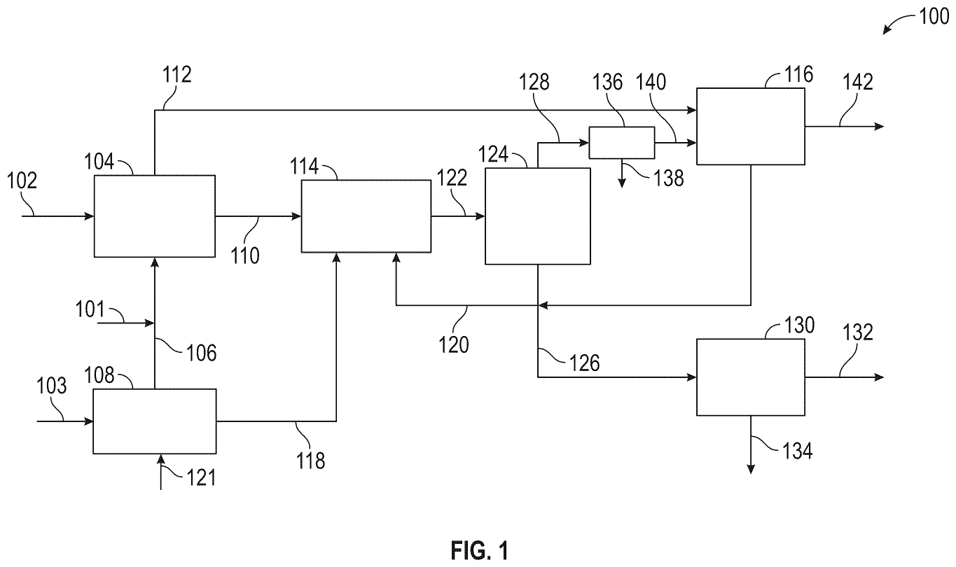

Referring now to FIG. 1, a schematic diagram illustrates one embodiment of a system 100 for producing methanol using syngas produced by an autothermal reactor 104, H2 produced by an electrolyzer 108 and a combined CO2 stream 120. A feed stream 102 using NG is fed to the autothermal reactor 104 with an O2 stream 106 produced by the electrolyzer 108 and a supplemental O2 stream 101 from an external source to meet the required chemical reaction stoichiometry. Through a process referred to as autothermal reforming (ATR), the autothermal reactor 104 produces a syngas stream 110 and a CO2 effluent stream 112 with a high concentration of post combustion CO2. The CO2 effluent stream 112 may be sent to a conventional amine absorption unit 116 for removal. The amine absorption unit 116 may include an absorber column and a desorber/stripper column that remove CO2 by a chemical absorption process, which exposes the CO2 effluent stream 112 to an aqueous amine solution. The syngas stream 110 is fed to a conventional methanol reactor 114 together with an H2 stream 118 from the electrolyzer 108 and the combined CO2 stream 120 from the amine absorption unit 116. Because the conversion of the combined CO2 stream 120 to methanol requires H2 beyond what is produced in the syngas stream 110, the electrolyzer 108 produces the additional H2 required for the conversion by using renewable power 121 to convert a demineralized water stream 103 into the H2 stream 118 and the O2 stream 106.

The methanol reactor 114 produces a methanol reactor output stream 122 comprising unreacted feed from the feed stream 102, syngas from the syngas stream 110, CO2 from the combined CO2 stream 120, and reaction products including water and methanol. The methanol reactor output stream 122 is sent to a separator 124 to be cooled, condensed and separated into a liquid stream 126 comprising a mixture of condensed raw methanol and water and a vapor stream 128 comprising a mixture of unreacted syngas, methane, methanol vapor, CO2, and water vapor. The liquid stream 126 is sent to a distillation column 130 where the condensed raw methanol is separated from the water to produce a purified methanol product stream 132 and a purified water by-product stream 134 that may be recycled. The vapor stream 128 is sent to a hydrogen separation unit 136, such as a pressure swing absorption (PSA) unit, which produces another H2 stream 138 and a CO2 off gas stream 140 with a high concentration of CO2, which may also include some syngas and unreacted hydrocarbon feeds. The another H2 stream 138 may be recycled back to methane reactor 114, and the CO2 off gas stream 140 may be sent to the amine absorption unit 116 for removal of CO2 by the chemical absorption process described above. CO2 is, therefore, removed from the CO2 off gas stream 140 and the CO2 effluent stream 112 by the amine absorption unit 116 to produce the combined CO2 stream 120 that is recycled back to the methanol reactor 114. Any syngas and unreacted hydrocarbon feeds in the CO2 off gas stream 140 are removed by the amine absorption unit 116 as a tail gas stream 142, which may be recycled back to the methanol reactor 114 or used as a fuel gas.

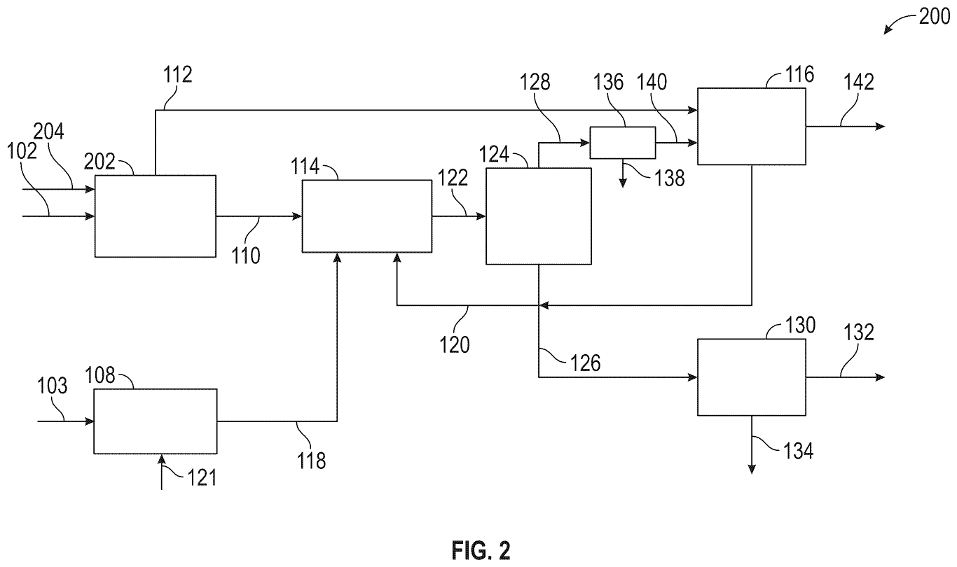

Referring now to FIG. 2, a schematic diagram illustrates one embodiment of a system 200 for producing methanol using syngas produced by a steam methane reformer 202, H2 produced by an electrolyzer 108 and a combined CO2 stream 120. A feed stream 102 using NG or other hydrocarbon (HC) feedstocks (e.g., LPG or naphtha) is fed to the steam methane reformer 202 with a steam stream 204. Through a process referred to as steam methane reforming (SMR), the steam methane reformer 202 uses a fired heater with a reforming catalyst inside reactor tubes with heat generated from burning a portion of the feed stream 102 in the fired heater to produce a syngas stream 110 and a CO2 effluent stream 112 with a high concentration of post-combustion CO2. The CO2 effluent stream 112 may be sent to a conventional amine absorption unit 116 for removal of CO2. The amine absorption unit 116 may include an absorber column and a desorber/stripper column that remove CO2 by a chemical absorption process, which exposes the CO2 effluent stream 112 to an aqueous amine solution. The syngas stream 110 is fed to a conventional methanol reactor 114 together with an H2 stream 118 from the electrolyzer 108 and the combined CO2 stream 120 from the amine absorption unit 116. Because the conversion of the combined CO2 stream 120 to methanol requires H2 beyond what is produced in the syngas stream 110, the electrolyzer 108 produces the additional H2 required for the conversion by using renewable power 121 to convert a demineralized water stream 103 into the H2 stream 118.

The methanol reactor 114 produces a methanol reactor output stream 122 comprising unreacted feed from the feed stream 102, syngas from the syngas stream 110, CO2 from the combined CO2 stream 120, and reaction products including water and methanol. The methanol reactor output stream 122 is sent to a separator 124 to be cooled, condensed and separated into a liquid stream 126 comprising a mixture of condensed raw methanol and water and a vapor stream 128 comprising a mixture of unreacted syngas, methane, methanol vapor, CO2, and water vapor. The liquid stream 126 is sent to a distillation column 130 where the condensed raw methanol is separated from the water to produce a purified methanol product stream 132 and a purified water by-product stream 134 that may be recycled. The vapor stream 128 is sent to a hydrogen separation unit 136, such as a pressure swing absorption (PSA) unit, which produces another H2 stream 138 and a CO2 off gas stream 140 with a high concentration of CO2, which may also include some syngas and unreacted hydrocarbon feeds. The another H2 stream 138 may be recycled back to methane reactor 114, and the CO2 off gas stream 140 may be sent to the amine absorption unit 116 for removal by the chemical absorption process described above. CO2 is, therefore, removed from the CO2 off gas stream 140 and the CO2 effluent stream 112 by the amine absorption unit 116 to produce the combined CO2 stream 120 that is recycled back to the methanol reactor 114. Any syngas and unreacted hydrocarbon feeds in the CO2 off gas stream 140 are removed by the amine absorption unit 116 as a tail gas stream 142, which may be recycled back to the methanol reactor 114 or used as a fuel gas.

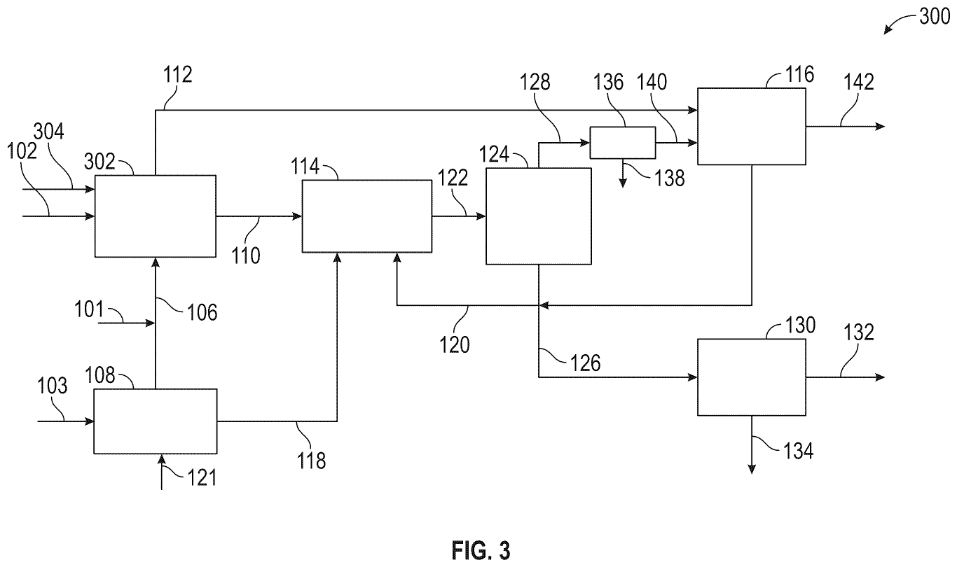

Referring now to FIG. 3, a schematic diagram illustrates one embodiment of a system 300 for producing methanol using syngas produced by a gasifier 302, H2 produced by an electrolyzer 108 and a combined CO2 stream 120. A feed stream 102 using a HC feedstock (e.g., naphtha, coal, pet-coke, biomass, and/or solid municipal waste) is fed to the gasifier 302 with a steam stream 304, an O2 stream 106 produced by the electrolyzer 108 and a supplemental O2 stream 101 from an external source to meet the required chemical reaction stoichiometry. Through a process referred to as gasification, the gasifier 302 produces a syngas stream 110 and a CO2 effluent stream 112 with a high concentration of post-combustion CO2. The gasifier 302 may include fluidized beds, moving beds, entrained flow systems and/or other conventional components/configurations. The CO2 effluent stream 112 may be sent to a conventional amine absorption unit 116 for removal. The amine absorption unit 116 may include an absorber column and a desorber/stripper column that remove CO2 by a chemical absorption process, which exposes the CO2 effluent stream 112 to an aqueous amine solution. The syngas stream 110 is fed to a conventional methanol reactor 114 together with an H2 stream 118 from the electrolyzer 108 and the combined CO2 stream 120 from the amine absorption unit 116. Because the conversion of the combined CO2 stream 120 to methanol requires H2 beyond what is produced in the syngas stream 110, the electrolyzer 108 produces the additional H2 required for the conversion by using renewable power 121 to convert a demineralized water stream 103 into the H2 stream 118 and the O2 stream 106.

The methanol reactor 114 produces a methanol reactor output stream 122 comprising unreacted feed from the feed stream 102, syngas from the syngas stream 110, CO2 from the combined CO2 stream 120, and reaction products including water and methanol. The methanol reactor output stream 122 is sent to a separator 124 to be cooled, condensed and separated into a liquid stream 126 comprising a mixture of condensed raw methanol and water and a vapor stream 128 comprising a mixture of unreacted syngas, methane, methanol vapor, CO2, and water vapor. The liquid stream 126 is sent to a distillation column 130 where the condensed raw methanol is separated from the water to produce a purified methanol product stream 132 and a purified water by-product stream 134 that may be recycled. The vapor stream 128 is sent to a hydrogen separation unit 136, such as a pressure swing absorption (PSA) unit, which produces another H2 stream 138 and a CO2 off gas stream 140 with a high concentration of CO2, which may also include some syngas and unreacted hydrocarbon feeds. The another H2 stream 138 may be recycled back to methane reactor 114, and CO2 off gas stream 140 may be sent to the amine absorption unit 116 for removal of CO2 by the chemical absorption process described above. CO2 is, therefore, removed from the CO2 off gas stream 140 and the CO2 effluent stream 112 by the amine absorption unit 116 to produce the combined CO2 stream 120 that is recycled back to the methanol reactor 114. Any syngas and unreacted hydrocarbon feeds in the CO2 off gas stream 140 are removed by the amine absorption unit 116 as a tail gas stream 142, which may be recycled back to the methanol reactor 114 or used as a fuel gas.

Other variations of the systems and methods described herein may include the production of ammonia using a syngas stream produced in an autothermal reactor in which a post-combustion CO2 stream is produced and fed to a dedicated methanol reactor with an H2 stream (in proper ratio to produce methanol) from an electrolyzer. An O2 stream from the electrolyzer is fed to the autothermal reactor or may be used in other O2 consuming processes. Other technologies may also be employed to separate CO2 such as, for example, the use of physical liquid absorbents or solid absorbents that may be used in pressure or vacuum swing absorption processes.

While the present disclosure has been described in connection with presently preferred embodiments, it will be understood by those skilled in the art that it is not intended to limit the disclosure of those embodiments. It is therefore contemplated that various alternative embodiments and modifications may be made to the disclosed embodiments without departing from the spirit and scope of the appended claims and equivalents thereof.

1. A system for producing methanol, which comprises:
a methanol reactor for producing a methanol reactor output stream, the methanol reactor comprising syngas from a syngas stream, hydrogen from a hydrogen stream and carbon dioxide from a recycled carbon dioxide stream;
a separator for separating the methanol reactor output stream into a vapor stream and a liquid stream;
a distillation column for separating the liquid stream into a methanol product stream and a water stream;
a hydrogen separation unit for separating the vapor stream into another hydrogen stream and a carbon dioxide off gas stream; and
an amine absorption unit for producing the recycled carbon dioxide stream and a tail gas stream, the anime absorption unit comprising carbon dioxide from the carbon dioxide off gas stream and post-combustion carbon dioxide from a carbon dioxide effluent stream.

2. (canceled)

3. The system of claim 2, further comprising:
an autothermal reactor for producing the carbon dioxide effluent stream and the syngas stream, the autothermal reactor comprising a natural gas feed from a feed stream and oxygen from an oxygen stream; and
an electrolyzer for producing the oxygen stream and the hydrogen stream, the electrolyzer comprising demineralized water from a water stream.

4. The system of claim 2, further comprising:
a steam methane reformer for producing the carbon dioxide effluent stream and the syngas stream, the steam methane reformer comprising a natural gas, naphtha or liquified petroleum gas feed from a feed stream and steam from a steam stream; and
an electrolyzer for producing the hydrogen stream, the electrolyzer comprising demineralized water from a water stream.

5. The system of claim 2, further comprising:
a gasifier for producing the carbon dioxide effluent stream and the syngas stream, the gasifier comprising a naphtha, coal, pet-coke, biomass or solid municipal waste feed from a feed stream, oxygen from an oxygen stream and steam from a steam stream; and
an electrolyzer for producing the oxygen stream and the hydrogen stream, the electrolyzer comprising demineralized water from a water stream.

6. The system of claim 2, wherein the methanol reactor output stream is in direct fluid communication with the separator.

7. The system of claim 2, wherein the recycled carbon dioxide stream from the amine absorption unit is in direct fluid communication with the methanol reactor.

8. The system of claim 2, wherein the vapor stream comprises a mixture of unreacted syngas, methane, methanol vapor, CO2, and water vapor.

9. The system of claim 2, wherein the liquid stream comprises a mixture of methanol and water.

10. The system of claim 1, wherein the methanol reactor output stream comprises syngas from the syngas stream, carbon dioxide from the recycled carbon dioxide stream, water, and methanol.

11. A method for producing methanol, which comprises:
introducing a syngas stream, a hydrogen stream and a recycled carbon dioxide stream into a methanol reactor for producing a methanol reactor output stream;
separating the methanol reactor output stream into a vapor stream and a liquid stream; separating the liquid stream into a methanol product stream and a water stream;
separating the vapor stream into another hydrogen stream and a carbon dioxide off gas stream, wherein the vapor stream is separated into the another hydrogen stream and the carbon dioxide off gas stream by a hydrogen separation unit and the recycled carbon dioxide stream and the tail gas stream are produced by an amine absorption unit comprising carbon dioxide from the carbon dioxide off gas stream and post-combustion carbon dioxide from a carbon dioxide effluent stream, and
producing the recycled carbon dioxide stream and a tail gas stream.

12. The method of claim 11, wherein the liquid stream is separated into the methanol product stream and the water stream by a distillation column.

13. (canceled)

14. (canceled)

15. The method of claim 14, wherein the carbon dioxide effluent stream and the syngas stream are produced by an autothermal reactor comprising a natural gas feed from a feed stream and oxygen from an oxygen stream, and the oxygen stream and the hydrogen stream are produced by an electrolyzer comprising demineralized water from a water stream.

16. The method of claim 14, wherein the carbon dioxide effluent stream and the syngas stream are produced by a steam methane reformer comprising a natural gas, naphtha or liquified petroleum gas feed from a feed stream and steam from a steam stream, and the hydrogen stream is produced by an electrolyzer comprising demineralized water from a water stream.

17. The method of claim 14, wherein the carbon dioxide effluent stream and the syngas stream are produced by a gasifier comprising a naphtha, coal, pet-coke, biomass or solid municipal waste feed from a feed stream, oxygen from an oxygen stream and steam from a steam stream, and the oxygen stream and the hydrogen stream are produced by an electrolyzer comprising demineralized water from a water stream.

18. The method of claim 11, wherein the methanol reactor output stream is in direct fluid communication with a separator.

19. The method of claim 14, wherein the recycled carbon dioxide stream from the amine absorption unit is in direct fluid communication with the methanol reactor.

20. The method of claim 11, wherein the methanol reactor output stream comprises syngas from the syngas stream, carbon dioxide from the recycled carbon dioxide stream, water, and methanol.
